SEQUENCE LISTING (15 sequences)

SEQ ID NO: 1 - DNA, 1182 bp, Homo sapiens
  CDS (1)..(1179)
atgcgcagcaccacgctcctggccctgctggcgctggtcttgctttacttggtgtctggtgccctggtgttccgggccctggagcagccccacgagcagcaggcccagagggagctgggggaggtccgagagaagttcctgagggcccatccgtgtgtgagcgaccaggagctgggcctcctcatcaaggaggtggctgatgccctgggagggggtgcggacccagaaaccaactcgaccagcaacagcagccactcagcctgggacctgggcagcgccttctttttctcagggaccatcatcaccaccatcggctatggcaatgtggccctgcgcacagatgccgggcgcctcttctgcatcttttatgcgctggtggggattccgctgtttgggatcctactggcaggggtcggggaccggctgggctcctccctgcgccatggcatcggtcacattgaagccatcttcttgaagtggcacgtgccaccggagctagtaagagtgctgtcggcgatgcttttcctgctgatcggctgcctgctctttgtcctcacgcccacgttcgtgttctgctatatggaggactggagcaagctggaggccatctactttgtcatagtgacgcttaccaccgtgggctttggcgactatgtggccggcgcggaccccaggcaggactccccggcctatcagccgctggtgtggttctggatcctgctcggcctggcttacttcgcctcagtgctcaccaccatcgggaactggctgcgagtagtgtcccgccgcactcgggcagagatgggcggcctcacggctcaggctgccagctggactggcacagtgacagcgcgcgtgacccagcgagccgggcccgccgccccgccgccggagaaggagcagccactgctgcctccaccgccctgtccagcgcagccgctgggcaggccccgatccccttcgccccccgagaaggctcagccgccttccccgcccacggcctcggccctggattatcccagcgagaacctggccttcatcgacgagtcctcggatacgcagagcgagcgcggctgcccgctgccccgcgcgccgagaggtcgccgccgcccaaatccccccaggaagcccgtgcggccccgcggccccgggcgtccccgagacaaaggcgtgccggtgtag

SEQ ID NO: 2 - protein, 393 aa, Homo sapiens
MRSTTLLALLALVLLYLVSGALVFRALEQPHEQQAQRELGEVREKFLRAHPCVSDQELGLLIKEVADALGGGADPETNSTSNSSHSAWDLGSAFFFSGTIITTIGYGNVALRTDAGRLFCIFYALVGIPLFGILLAGVGDRLGSSLRHGIGHIEAIFLKWHVPPELVRVLSAMLFLLIGCLLFVLTPTFVFCYMEDWSKLEAIYFVIVTLTTVGFGDYVAGADPRQDSPAYQPLVWFWILLGLAYFASVLTTIGNWLRVVSRRTRAEMGGLTAQAASWTGTVTARVTQRAGPAAPPPEKEQPLLPPPPCPAQPLGRPRSPSPPEKAQPPSPPTASALDYPSENLAFIDESSDTQSERGCPLPRAPRGRRRPNPPRKPVRPRGPGRPRDKGVPV

SEQ ID NO: 3 - DNA, 27 bp, Artificial Sequence
  Description of Artificial Sequence Primer
agaattcgcgccatgcgcagcaccacg

SEQ ID NO: 4 - DNA, 27 bp, Artificial Sequence
  Description of Artificial Sequence Primer
tttctcgaggcccggccagggatcctg

SEQ ID NO: 5 - DNA, 20 bp, Artificial Sequence
  Description of Artificial Sequence Primer
acccagtggaggagcccttc

SEQ ID NO: 6 - DNA, 21 bp, Artificial Sequence
  Description of Artificial Sequence Primer
gaggcccggccagggatcctg

SEQ ID NO: 7 - DNA, 20 bp, Artificial Sequence
  Description of Artificial Sequence Synthetic oligonucleotide
ccaggctgccagctggactg

SEQ ID NO: 8 - DNA, 20 bp, Artificial Sequence
  Description of Artificial Sequence Primer
ctcagtgctcaccaccatcg

SEQ ID NO: 9 - DNA, 21 bp, Artificial Sequence
  Description of Artificial Sequence Primer
gaggcccggccagggatcctg

SEQ ID NO: 10 - DNA, 195 bp, Homo sapiens
atgcgcagcaccacgctcctggccctgctggcgctggtcttgctttacttggtgtctggtgccctggtgttccgggccctggagcagccccacgagcagcaggcccagagggagctgggggaggtccgagagaagttcctgagggcccatccgtgtgtgagcgaccaggagctgggcctcctcatcaaggtgcgt

SEQ ID NO: 11 - DNA, 155 bp, Homo sapiens
cctccttctgcaccttgtcctgcaggaggtggctgatgccctgggagggggtgcggacccagaaaccaactcgaccagcaacagcagccactcagcctgggacctgggcagcgccttctttttctcagggaccatcatcaccaccatcggtgggg

SEQ ID NO: 12 - DNA, 178 bp, Homo sapiens
cttcctgcatcgcccctctgcccaggctatggcaatgtggccctgcgcacagatgccgggcgcctcttctgcatcttttatgcgctggtggggattccgctgtttgggatcctactggcaggggtcggggaccggctgggctcctccctgcgccatggcatcggtcacattggtgagc

SEQ ID NO: 13 - DNA, 232 bp, Homo sapiens
tcgtgcctactgccccatcccgcagaagccatcttcttgaagtggcacgtgccaccggagctagtaagagtgctgtcggcgatgcttttcctgctgatcggctgcctgctctttgtcctcacgcccacgttcgtgttctgctatatggaggactggagcaagctggaggccatctactttgtcatagtgacgcttaccaccgtgggctttggcgactatgtggccggtgagg

SEQ ID NO: 14 - DNA, 171 bp, Homo sapiens
tctaccttccctggtgggtacccaggcgcggaccccaggcaggactccccggcctatcagccgctggtgtggttctggatcctgctcggcctggcttacttcgcctcagtgctcaccaccatcgggaactggctgcgagtagtgtcccgccgcactcgggcagaggtaggc

SEQ ID NO: 15 - DNA, 406 bp, Homo sapiens
agggctgctttccctctccgtgcagatgggcggcctcacggctcaggctgccagctggactggcacagtgacagcgcgcgtgacccagcgagccgggcccgccgccccgccgccggagaaggagcagccactgctgcctccaccgccctgtccagcgcagccgctgggcaggccccgatccccttcgccccccgagaaggctcagccgccttccccgcccacggcctcggccctggattatcccagcgagaacctggccttcatcgacgagtcctcggatacgcagagcgagcgcggctgcccgctgccccgcgcgccgagaggtcgccgccgcccaaatccccccaggaagcccgtgcggccccgcggccccgggcgtccccgagacaaaggcgtgccggtgtag

(19) United States
(12) Patent Application Publication
(10) Pub. No.: US 2003/0049697 A1
(43) Pub. Date: Mar. 13, 2003

(54) Family of mechanosensitive human potassium channels activated by polyunsaturated fatty acids and their use

(75) Inventors: Michel Lazdunski, Nice (FR); Florian Lesage, Nice (FR); Francois Maingret, Antibes (FR)

Correspondence Address: SCHNADER HARRISON SEGAL & LEWIS, LLP, 1600 MARKET STREET, SUITE 3600, PHILADELPHIA, PA, 19103

(73) Assignee: CENTRE NATIONAL DE LA RECHERCHE SCIENTIFIQUE, C.N.R.S., PARIS (FR)

(21) Appl. No.: 10/243,035

(22) Filed: Sep. 13, 2002

Related U.S. Application Data
(63) Continuation of application No. PCT/FR01/00758, filed on Mar. 14, 2001.

(30) Foreign Application Priority Data
Mar. 14, 2000 (FR) 00/03264

(51) Int. Cl. G01N 33/567

(52) U.S. Cl. 435/7.21

(57) ABSTRACT

A mechanosensitive human potassium channel, referred to as “hTRAAK” for human TWICK-Related AA-Activated K⁺ channel, which is activated by polyunsaturated fatty acids as well as by the neuroprotective agent riluzole. The properties of the channels of the TRAAK family as well as their tissue distribution give these channels a primordial role in the transport of potassium in a large number of cell types.

### RELATED APPLICATION

This application is a continuation of PCT/FR01/00758 filed Mar. 14, 2001, which claims benefits from French Application No. 00/03264 filed Mar. 14, 2000.

### FIELD OF INVENTION

This invention concerns a new class of mechanosensitive potassium channels activated by polyunsaturated fatty acids. The invention is based on the discovery of a new mechanosensitive human potassium channel, referred to as “hTRAAK” for human TWICK-Related AA-Activated K⁺ channel, which is activated by polyunsaturated fatty acids as well as by the neuroprotective agent riluzole. The properties of the channels of the TRAAK family as well as their tissue distribution give these channels a primordial role in the transport of potassium in a large number of cell types.

### BACKGROUND

The potassium channels are ubiquitous proteins and their exceptional functional diversity makes them ideal candidates for a large number of biological processes. They are involved notably in the regulation of neuronal and muscular excitability, cardiac rhythm and hormone secretion.

To date, six members of this family have been cloned: TWIK-1, TWIK-2, TASK-1, TASK-2, TREK-1 and TRAAK (Chavez et al., 1999; Duprat et al., 1997; Fink et al., 1996; Fink et al., 1998; Lesage et al., 1996; Reyes et al., 1998). Despite an overall similar structure, the sequence identity among these channels is weak (less than 30%). TWIK-1 and TWIK-2 are weak inward rectifying K⁺ channels. TASK-1 and TASK-2 are outward rectifying K⁺ channels sensitive to extracellular variations in pH in a narrow physiological range. TREK-1, another outward rectifying K⁺ channel, is activated by membranal stretching, polyunsaturated fatty acids, intracellular acidosis and inhaled anesthetics (Maingret et al., 1999 (b); Patel et al., 1999; Patel et al., 1998). These two-pore K⁺ channels have an extensive tissue distribution. TRAAK, the second cloned mechanosensitive K⁺ channel activated by polyunsaturated acids, is the only one that is expressed exclusively in the central nervous system (Fink et al., 1998; Maingret et al., 1999 (a)).

Applications using the TRAAK potassium channels of the mouse are disclosed in French patent application 98/02725.

### SUMMARY OF THE INVENTION

This invention relates to mechanosensitive potassium channels activated by polyunsaturated fatty acids. The new mechanosensitive human potassium channel, referred to as “hTRAAK” for human TWICK-Related AA-Activated K⁺ channel, is activated by polyunsaturated fatty acids as well as by the neuroprotective agent riluzole. The invention also relates to methods of screening, diagnosis, prevention and treatment associated with “hTRAAK”, as well as nucleic acid molecules, vectors and cells associasted with “hTRAAK”.

### BRIEF DESCRIPTION OF THE DRAWINGS

Selected advantages and characteristics of the invention will become apparent upon reading the examples below which report the research activities that led to the identification and characterization of these mechanosensitive potassium channels which are activated by fatty acids. These examples refer to the attached sequences and drawings in which:

FIG. 1A represents the topology of hTRAAK.

FIG. 1B and SEQ ID No: 1 represent the nucleotide sequence of the cDNA of hTRAAK. FIG. 1B and SEQ ID No: 2 represent the amino acid sequence of the coding sequence.

FIG. 2 represents the idiogram of the G bands of human chromosome 11q and the localization of the hTRAAK gene in relation to the markers localized in a Genebridge 4 RH panel.

FIG. 3 represents the RT-PCR analysis of the distribution of hTRAAK in adult human tissues.

FIG. 4 generally shows the biophysical properties of hTRAAK recorded using the imposed voltage technique on COS cells transfected with a vector expressing hTRAAK.

FIG. 4A particularly shows that the hTRAAK current does not have an apparent threshold of voltaic activation, is independent of time and cannot be activated.

FIG. 4B shows the I-V curve, the slope of the restriction curve is 58.6±0.6 mV by changing by a factor of 10 the exterior concentration of K⁺ (n=6).

FIG. 4C shows the directional change of the hTRAAK current in a physiological gradient (5 mM ext. K⁺).

FIG. 4D shows the properties of an hTRAAK channel alone.

FIG. 5 shows the effect of arachidonic acid (AA) on the hTRAAK channel expressed in the transfected COS cells.

FIG. 5A shows the activity of human TRAAK potentiated by 10 μm of arachidonic acid (AA) in the whole cell configuration (630±101% at 0 mV, n=19).

FIG. 5B shows the effect of arachidonic acid (AA) on the hTRAAK channel expressed in the transfected CO cells when the exterior Na⁺ is replaced by K⁺.

FIG. 5C shows the current induced by AA observed in an outside-out configuration.

FIG. 5D shows the current recorded for an hTRAAK channel alone in an inside-out configuration at −50 mV and 50 mV

### DETAILED DESCRIPTION

This invention is based on the discovery and cloning of a new channel designated “hTRAAK” which is a member of the family of TWIK channels. The gene coding this channel shares the functional properties of its murine equivalent (Fink et al., 1998; Maingret et al., 1999 (a)) and also is principally expressed in the neuronal tissues.

The discovery of this new class of potassium channels and the heterologous expression of these channels has a number of utilities to those of ordinary skill in the art. For example, this discovery provides new research tools for screening drugs that are capable of modulating the activity of the potassium channels. This discovery also provides methods of preventing and/or treating diseases involving these channels including but not limited to epilepsy, cardiac pathologies (arrhythmias) and vascular diseases, neurodegenerative diseases, especially those associated with ischemia and anoxia, endocrine diseases associated with defective hormone secretion, muscle diseases and retinal pathologies, as well as pharmaceutical compositions to effect such methods.

Thus, one aspect of the invention is a purified protein constituting a mechanosensitive human potassium channel activated by polyunsaturated fatty acids, especially arachidonic acid, and by riluzole. More specifically, the invention pertains to the protein constituting the human TRAAK channel, the amino acid sequence of which is represented in SEQ ID No: 2 or a variant which is a functionally equivalent derivative of this protein.

Such variants include those with a sequence comprising a modification and/or a suppression and/or an addition of one or more amino acid residues, so long as this modification and/or suppression and/or addition does not modify the properties of the hTRAAK channel. Such variants can be readily determined by those of ordinary skill in the art using the techniques described in the examples presented below which enable demonstration of the biophysical and pharmacological properties of the hTRAAK channel.

Polyclonal or monoclonal antibodies directed against at least one protein constituting an ionic channel of the invention can be prepared by conventional methods described in the literature. These antibodies are useful for detecting the presence of the ionic channels of the invention in various human and animal tissues. However, because of their specificity, they can also find therapeutic applications for the in vivo inhibition or activation of an hTRAAK channel and/or its derivatives.

This invention also relates to an isolated and/or purified nucleic acid molecule comprising or constituted by a nucleic sequence coding for a protein constituting a mechanosensitive human potassium channel activated by polyunsaturated fatty acids, especially arachidonic acid, and by riluzole. More specifically, the invention pertains to a nucleic acid molecule comprising at least one sequence coding for the protein constituting the hTRAAK channel, the amino acid sequence of which is SEQ ID No: 2 or for a variant which is a functionally equivalent derivative of this protein. A DNA molecule comprising the sequence coding for the hTRAAK protein is SEQ ID No: 1. The invention also pertains to its complementary sequence.

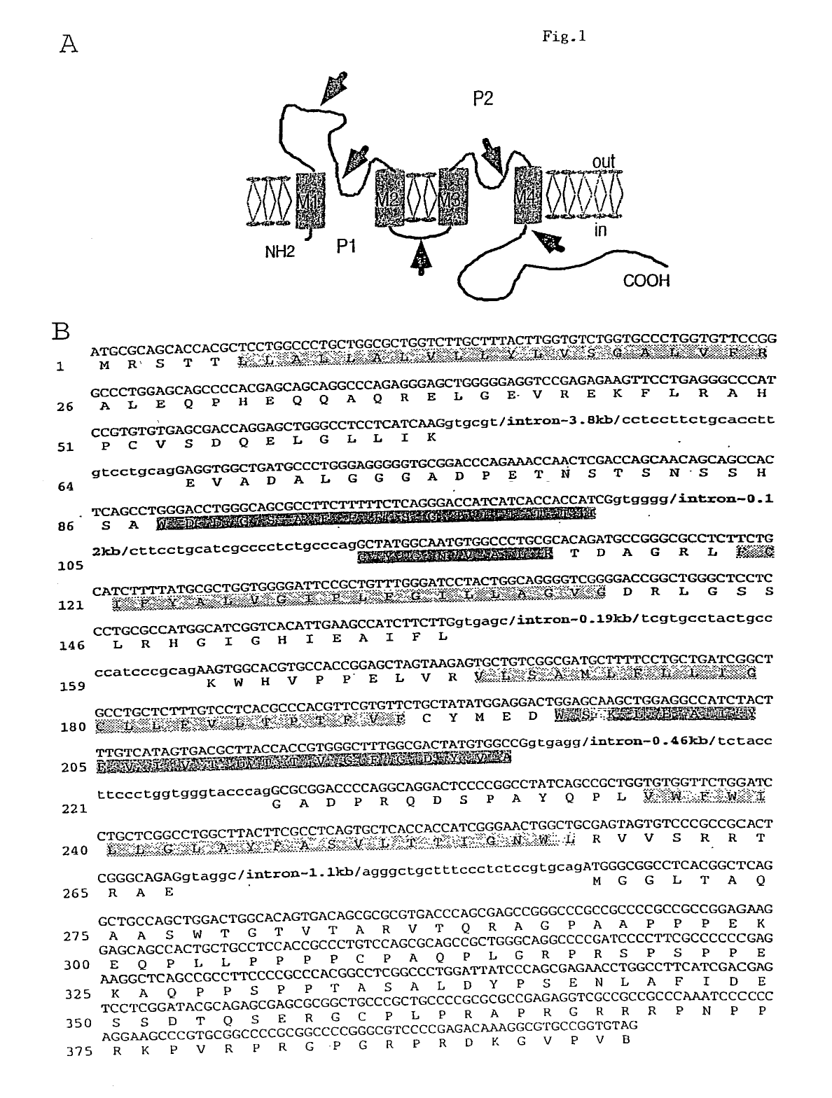

The FIG. 1B nucleotide fragments have been assigned SEQ ID Nos. 10-15, respectively, in order of appearance.

The invention also pertains to a vector comprising at least one of the preceding nucleic acid molecules, advantageously associated with suitable control sequences, as well as a process for the production or expression in a cellular host of a protein constituting an ionic channel according to the invention. The preparation of these vectors as well as the production or expression in a host of the channels of the invention can be implemented by molecular biology and genetic engineering techniques which are well known to those of ordinary skill in the art.

As an example, a process for the production of a protein constituting a cationic channel according to the invention comprises:

transferring a nucleic acid molecule of the invention or a vector containing said molecule into a cellular host,

culturing the cellular host under conditions enabling production of the protein constituting the potassium channel, and

isolating by any suitable means the proteins constituting the potassium channels of the invention.

As an example, a process for the expression of an ionic channel according to the invention comprises:

transferring a nucleic acid molecule of the invention or a vector containing the molecule into a cellular host, and

culturing the cellular host under conditions enabling expression of the potassium channels.

The cellular host employed in the preceding processes can be selected from among prokaryotes or eukaryotes and especially from among bacteria, yeasts, and mammal, plant or insect cells.

The vector employed is selected on the basis of the host into which it will be transferred. All vectors such as plasmids can be employed.

Thus, the invention also pertains to cellular hosts and more specifically transformed cells expressing the potassium channels exhibiting the properties and structure of the type of hTRAAK channel cells obtained in accordance with the preceding processes. These cells are useful for screening substances capable of modulating the TRAAK channel currents. This screening is implemented by bringing into contact variable quantities of a substance to be tested with cells expressing the channels of the invention, then measuring by any suitable means the possible effects of said substance on the potassium currents of said channels. Electrophysiological techniques also make these studies possible and are also the object of the invention when employed with hTRAAK channels or their variants. This screening process makes it possible to identify drugs that can modulate the activity of the potassium channels of the invention and, thus, might be able to prevent or treat the diseases in which these channels are involved. These substances and their use as drugs, isolated and detected by means of the above process, are also part of the invention.

More specifically, the invention thus pertains to a chemical or biological substance capable of modifying the currents of a potassium channel for preparation of a drug that is useful for the prevention or treatment of diseases of the heart or nervous system in human or animal subjects, such as cardiac pathologies (arrhythmias) and vascular diseases, neurodegenerative diseases, especially those associated with ischemia and anoxia, endocrine diseases associated with defective hormone secretion and muscle diseases.

A nucleic acid molecule coding for a protein constituting an hTRAAK channel or a derivative thereof, or a vector comprising this nucleic acid molecule or a cell expressing TRAAK channels are also useful for the preparation of transgenic animals. These can be animals that overexpress the channels, but more especially knock-out animals, e.g., animals having a deficiency in these channels. These transgenic animals are prepared by methods which are known to those of ordinary skill in the art, and allow preparation of live models for studying the animal pathologies associated with the TRAAK channels.

These transgenic animals as well as the previously described cellular hosts are useful as models for studying the pathologies associated with these mechanosensitive potassium channels which are activated by polyunsaturated fatty acids either because they overexpress the potassium channels of the hTRAAK channel type or because they have a deficiency in these potassium channels.

The invention also pertains to the in vitro diagnosis of pathologies in humans and/or animals which could involve the mechanosensitive potassium channel activated by polyunsaturated fatty acids, especially arachidonic acid, and by riluzole. This in vitro diagnosis can be performed by any means employing a procedure for the detection or localization in a biological sample of the potassium channel or the gene coding for the potassium channel.

These detection procedures can use either polyclonal or monoclonal antibodies directed against the protein, against a variant of the protein or against at least one fragment of the protein constituting the ionic channel, or one or more nucleotide probes capable of hybridizing with the gene coding for the potassium channel or with a variant of it or with at least one fragment of it.

Thus, the invention concerns the use of the previously described polyclonal or monoclonal antibodies or of their fragments for the detection of pathologies in humans and/or animals. This is achieved by detecting the mutation and/or suppression and/or addition of at least one amino acid in the protein constituting the potassium channel according to the invention or a variant thereof. The invention also pertains to the use of the nucleic acid sequences according to the invention or the oligonucleotides stemming from them for the detection of pathologies in humans and/or animals. This is achieved by detecting the mutation and/or suppression and/or addition of at least one nucleotide in the nucleotide sequences.

These in vitro detection procedures can be applied to the detection of any pathology involving the potassium channels of the invention, such as cardiac and vascular pathologies, pathologies of the nervous system associated with ischemia and/or anoxia, pathologies of the spinal cord, endocrine pathologies associated with anomalies in the secretion of hormones, muscle pathologies or pathologies of the retina in human or animal subjects.

In addition, a protein constituting a neuronal ionic hTRAAK channel can also be useful for the manufacture of drugs intended to treat or prevent the diseases in which these channels are involved. The invention, thus, also pertains to pharmaceutical compositions comprising as an active agent at least one of these proteins possibly combined with a physiologically acceptable vehicle.

In fact, the nucleic acid molecules of the invention or the cells transformed by the molecules are suitable for use in gene therapy strategies to compensate for an hTRAAK channel deficiency at the level of one or more tissues of a patient. The invention, thus, also pertains to a drug comprising the nucleic acid molecules of the invention or cells transformed by said molecules for the prevention or treatment of diseases in which the hTRAAK channels or their derivatives are involved.

I—Identification, Primary Structure and Tissue Distribution of hTRAAK

Studies of the DNA bases using the BLAST sequence alignment program (Altschul et al., 1990) provided identification of human sequences restricted to a simple genomic contig. The analysis of these sequences suggested the presence of introns and exons forming a gene coding a two-pore K⁺ channel. The oligonucleotides were deduced from the potential exon sequences and used for PCR amplification of a DNA fragment containing the corresponding open reading phase (ORF) from brain cDNA. This ORF is 1182 nucleotides in length and codes a polypeptide of 393 amino acids (FIG. 1B, SEQ ID No: 2). This protein is close to the mouse TRAAK channel with 82% identity and 88% homology. This level of homology, along with the tissue distribution and the conserved functional properties which are shown below, indicates that the new channel is a homologue of the murine TRAAK. FIG. 1B shows the cDNA sequence of TRAAK and the genomic organization in humans. The ORF is composed of six exons. The transmembranal segment M1 is coded by exon 1, M2 by exon 3, M3 by exon 4 and M4 by exon 5. The second exon codes the C-terminal part of the interdomain M1P1 and the sixth the large C-terminal part of the channel (FIGS. 1A and 1B).

The introns are short with the exception of the first one which is long at more than 3.8 Kb (FIG. 1B). A gene coding another two-pore mammalian K⁺ channel, TWIK-1, has already been characterized (Arrighi et al., 1998). The organizations of TRAAK and TWIK-1 are rather different because TWIK-1 contains only three exons separated by two broad introns. Nevertheless, a characteristic common to these two genes is the presence of an intron in the first pore domain P1. The intron site is between the first and second nucleotides of the codon for the first glycine residue of the signature GYG sequence of the pore (Arrighi et al., 1998). An intron in the same position was found in 20 genes from among the 36 genes examined which code two-pore K⁺ channels in the nematode Caenorhabditis elegans (Wang et al., 1999). The significance of the conserved position of this intron is not known. Nevertheless, it should be noted that this intron was conserved in mammals in which it could possibly have the same role as in nematodes.

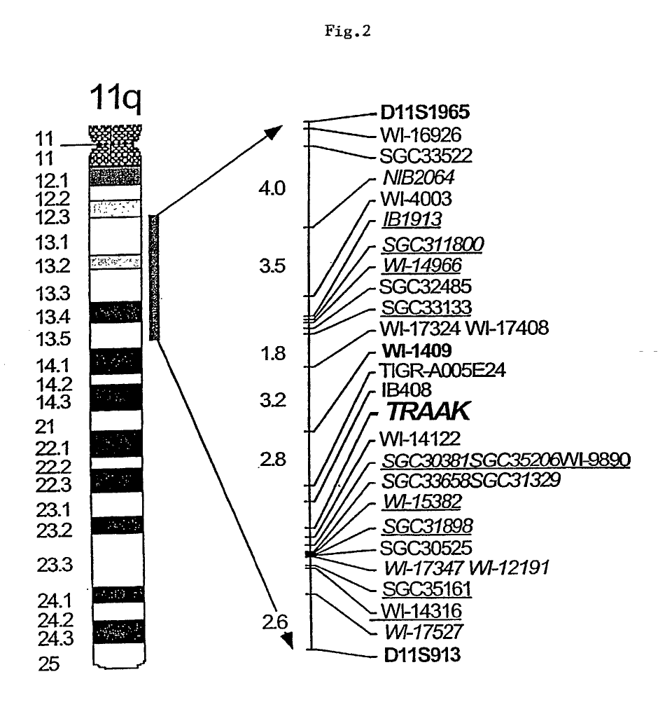

The chromosomal characterization of human TRAAK was performed by an analysis of radiation hybrid panels. As shown in FIG. 2, the gene coding hTRAAK is found on chromosome 11q and is telomeric at 5.34 cRays of the marker WI-1409 (logarithm of the score >21). Although hybrid radiation maps are not linked to cytogenetic maps, the most probable localization of the hTRAAK gene is 11q13. KCNK7, which contains a K⁺ channel with two P domains was localized on chromosome 11q13 telomeric at 6.4 cRays of WI-1409. This suggests that hTRAAK and KCNK7 are very close to each other (Salinas et al., 1999).

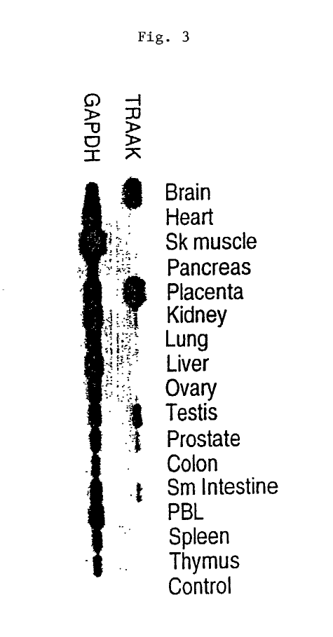

The expression of hTRAAK in different adult human tissues was studied by RT-PCR analysis. As shown in FIG. 3, TRAAK is expressed at the highest levels in the brain and the placenta. Only very weak signals were obtained in the testicles, the small intestine, the prostate and the kidney. hTRAAK was not detected in the mouse placenta (Fink et al., 1998). The reason for these contradictory findings is not known. In situ hybridization (Fink et al., 1998) and immunologic hybridization (Reyes et al., 2000) have shown that murine TRAAK is specifically expressed in the neuronal cells. The tissue distribution shown in FIG. 3 suggests that hTRAAK has the same restricted pattern of expression in humans.

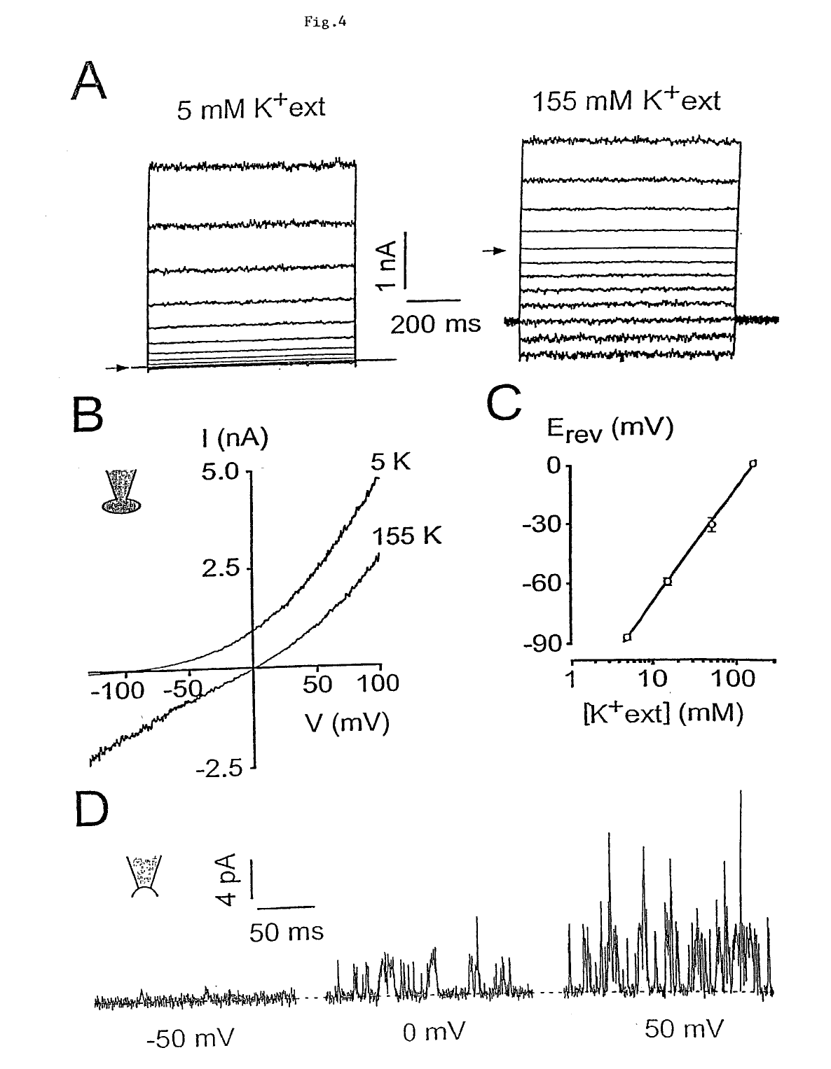

Electrophysiological experiments were performed in COS cells transfected in a temporary manner. The hTRAAK current does not have an apparent voltaic threshold of activation, is independent of time and cannot be activated (FIG. 4A). The I-V curve is outward rectifying and tumultuous at positive potentials (FIGS. 4A and 4B). In a physiological gradient (5 mM ext. K⁺), the hTRAAK current changes direction at the predicted equilibrium value for K⁺ (−87.1±1.2 mV, n=6). When the exterior Na⁺ is replaced by K⁺, the direction changing potential closely follows the equilibrium value of K⁺ (FIG. 4C). The slope of the restriction curve is 58.6±0.6 mV by changing by a factor of 10 the exterior concentration of K⁺ (n=6) which is in agreement with the Nernst's equation for a channel selective for K⁺. In a symmetrical gradient (155 mM exterior K⁺), the I-V curve is almost linear (FIGS. 4A and 4B) and changes direction at 0.8±1.1 mV (n=6).

The pharmacological properties of hTRAAK were studied in the whole cell configuration. hTRAAK is insensitive to the conventional blocking agents of the K⁺ channels quinidine (100 μm), 4AP (3 mM), TEA (10 mM), barium (1 mM) and glibenclamide (10 μM). It has been shown that certain members of the family of K⁺ channels with two P domains (TREK-1 and TASK-1) are opened by volatile general anesthetics (Patel et al., 1999). We, therefore, also investigated the effect of chloroform on hTRAAK. The application of chloroform (0.8 mM, n=8) has no effect on the channel's activity. The properties of an hTRAAK channel alone are illustrated in FIG. 1D. At the microscopic level, the hTRAAK remains outward rectifying and is characterized by an oscillating behavior.

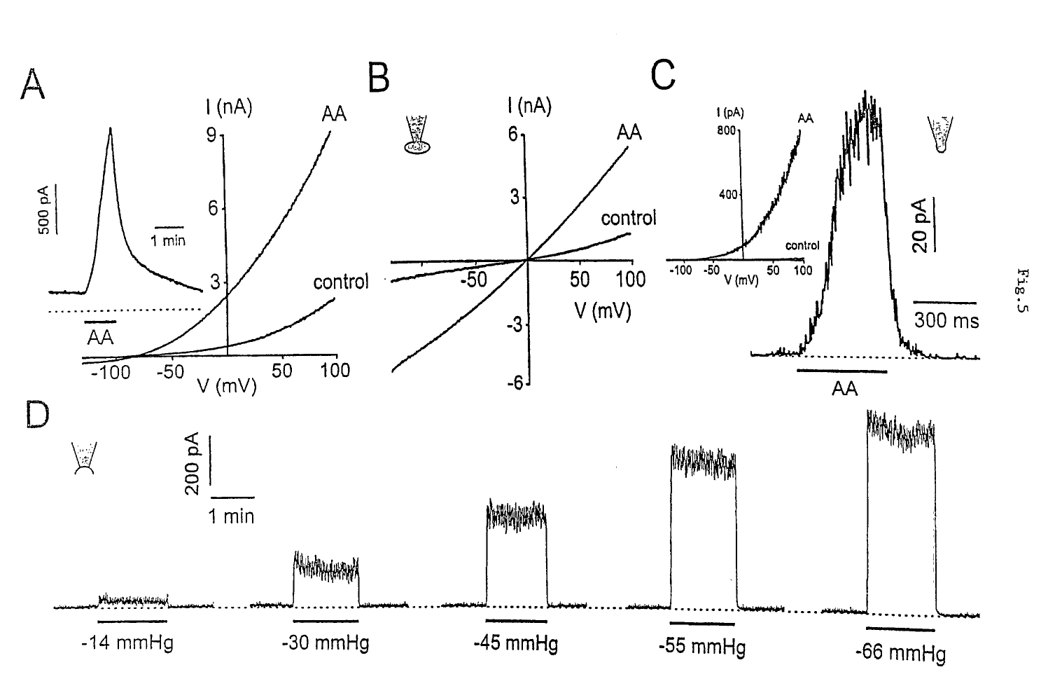

FIGS. 5A and 5B show that the activity of human TRAAK, like murine TRAAK (Fink et al., 1998) is potentiated by 10 μm of arachidonic acid (AA) in the whole cell configuration (630±101% at 0 mV, n=19). This activation is completely reversible by means of washing (FIG. 5A, box). Under physiological conditions, the current induced by AA is outward rectifying and changes direction at 80.6±0.9 mV, n=7 (FIG. 5A). When the exterior Na⁺ is replaced by K⁺, the current becomes linear and the direction change potential is −0.6±0.8 mV, n=7 (FIG. 5B). hTRAAK is also activated by the polyunsaturated acid decosahexaenoate (10 μM, n=3), but is insensitive to the saturated fatty acids myristate, palmitate, stearate and arachinate (10 μM, n=6 to 8). Moreover, the AA derivatives with an alcohol or a methyl ester substituted by the carboxyl function are inactive (n=5). The stimulation by AA of hTRAAK remains then when the patch is excised (FIG. 5C). The current induced by AA observed in an outside-out configuration is outward rectifying and changes direction at the reverse potential of K⁺ (FIG. 5C, box). We showed that the K⁺ channels with two P domains activated by polyunsaturated fatty acids (mTREK-1 and MTRAAK) are mechanosensitive K⁺ channels. In fact, the opening of the channel is mediated by a deformation of the membrane (Maingret et al., 1999 (a); Patel et al., 1998). FIG. 5D illustrates the mechanical sensitivity of hTRAAK. In the reverse patch configuration, the activity of the channel is almost absent at atmospheric pressure. Application of negative pressure opens the channels in a dose-dependent manner. Taken together, these results show that human TRAAK shares the same biophysical and pharmacological properties as its murine homologue (Fink et al., 1998; Maingret et al., 1999 (a); Patel et al., 1999).

II—Cloning hTRAAK cDNA

The sequences of K⁺ channels with two P domains were used to search for homologues in the public DNA databases using the BLAST program (Altschul et al., 1990). This led to identification of a genomic sequence (Genbank accession number AC005848) which exhibited significant similarities with murine TRAAK. Two oligonucleotides were selected from this genomic sequence corresponding to the equivalent sequences flanking the first initiation codon and the stop codon of mTRAAK: sense strand: 5′-AGAATTCGCGCCATGCGCAGCACCACG-3′ (SEQ ID No: 3) and antisense strand: 5′-TTTCTCGAGGCCCGGCCAGGGATCCTG-3′ (SEQ ID No: 4) introducing the restriction sites EcoRI and XhoI, respectively. The entire coding sequence was amplified from human brain cDNA using these primers and a DNA polymerase with a low error rate then subcloned in a pIRES-CD8 vector to yield pIRES-CD8.hTRAAK. Inserts from independent PCR-ligation experiments were sequenced on the two strands and found to be identical.

III—Chromosomal Mapping

The Genebridge 4 RH DNA panel (Research Genetics) was screened by PCR using primers deduced from intron 5 (sense primer: 5′-ACCCAGTGGAGGAGCCCTTC-3′) (SEQ ID No: 5) and exon 6 (antisense primer: 5′-GAGGCCCGGCCAGGGATCCTG-3′) (SEQ ID No: 6). The PCR conditions were 39 cycles of 30 s at 94° C., 30 s at 55° C. and 30 s at 72° C. The PCR products were separated by agarose electrophoresis then transferred onto charged nylon membranes. The blots were analyzed with an oligonucleotide labeled with P32 5′-CCAGGCTGCCAGCTGGACTG-3′ (SEQ ID No: 7). The results were analyzed using the RH-MAPPER program at the Whitehead Institute.

IV—RT-PCR Experiments

cDNA of various tissue types (Clontech) were used as master according to the supplier's protocol. The primer sequences were for the sense primer: 5′-CTCAGTGCTCACCACCATCG-3′ (SEQ ID No: 8) (exon 5) and for the antisense primer: 5′-GAGGCCCGGCCAGGGATCCTG-3′ (SEQ ID No: 9) (exon 6). The PCR conditions were 34 cycles of 30 s at 94°, 30 s at 55° C. and 1 min at 72° C. The PC products were separated, transferred and analyzed as described for the chromosomal mapping.

V—Cell culture and transfection

The COS-7 cells were maintained in Eagle medium as modified by Dulbecco supplemented with 10% fetal calf's serum. The plasmid pIRES-cD8-hTRAAK was transfected using the conventional DEAE dextran procedure. The positive cells were visualized 48 h after transfection using the method of beads covered by the anti-CD8 antibody (Maingret et al., 1999 (a); Maingret et al., 1999 (b)).

VI—Electrophysiology

For the experiments on whole cells and the outside-in experiments, the pipette solution (INT) contained 150 mM KCl, 3 nM MgCl₂, 5 mM EGTA and 10 mM HEPES, pH adjusted with KOH. The bath solution (EXT) contained 150 mM NaCl, 5 mM KCl, 3 mM MgCl₂, 1 mM CaCl₂ and 10 mM HEPES, pH 7.4 adjusted with NaOH. For the inside-out experiments, the solution in the pipette was EXT and the bath solution was INT. The EXT solution rich in K⁺ contained 150 mM KCl in place of 150 mM NaCl. To study the selectivity of the ions, the relationships between the current and the voltage were obtained at different ext. K⁺ concentrations. For each concentration, NaCl was replaced in the EXT solution by equimolar KCl.

All of the products were obtained from Sigma. The fatty acids were dissolved in ethanol at the concentration of 100 mM, put under argon and stored at 20 C for one week. The mechanical stimulation was applied by a system generating the pressure in open loop and controlled at the level of the pipette during the experiment by a calibrated pressure sensor.

### BIBLIOGRAPHIC REFERENCES

The subject matter of the below listed References is hereby incorporated herein by reference.

Altschul et al., 1990 “Basic local alignment search tool”, J. Mol. Biol., 215, 403-10.

Arrighi et al., 1998, “Structure, chromosome localization and tissue distribution of the mouse twik K+ channel gene”, FEBS Lett., 425, 310-6.

Chavez et al., 1999, “TWIK-2, a new weak inward rectifying member of the tandem pore domain potassium channel family”, J. Biol. Chem., 274, 7887-92.

Duprat et al., 1997, “TASK, a human background K+ channel to sense external pH variations near physiological pH”, EMBO J., 16, 5464-71.

Fink et al., 1996, “Cloning, functional expression and brain localization of a novel unconventional outward rectifier K+ channel”, EMBO J., 15, 6854-62.

Fink et al., 1998, “A neuronal two P domain K+ channel activated by arachidonic acid polyunsaturated fatty acid”, EMBO J., 17, 3297-308.

Lesage et al., 1996, “TWIK-1, a ubiquitous human weakly inward rectifying K+ channel with a novel structure”, EMBO J., 15, 1004-11.

Maingret et al., 1999(a), “TRAAK is a neuro nal mechano-gated K+ channel”, J. Biol. Chem., 274, 1382-7.

Maingret et al., 1999(b), “Mechano- or acid stimulation, two interactive modes of activation of the TREK-1 potassium channel”, J. Biol. Chem., 274, 26691-6.

Patel et al., 1999, “Inhalational anaesthetics activate two-pore domain background K+ channels”, Nature Neurosci., 2, 422-6.

Patel et al., 1998, “A mammalian two pore domain mechano-gated S-like K+ channel”, EMBO J., 17, 4283-90.

Reyes et al., 1998, “Cloning and expression of a novel pH sensitive two pore domain potassium channel from human kidney”, J. Biol. Chem., 273, 30863-9.

Reyes et al., 2000, “Immunolocalization of the arachidonic acid mechanosensitive baseline TRAAK potassium channel in the nervous system”, Neuroscience, 95, 893-901.

Salinas et al., 1999, “Cloning of a new mouse two-P domain channel subunit and a human homologue with a unique pore structure”, J. Biol. Chem., 274, 11571-60.

Wang et al., 1999, “Genomic organization of nematode 4TM K+ channels”, Ann. NY Acad Sci., 868, 286-303.

1. A method of screening for a substance comprising:
contacting a sample containing the substance which prevents and/or treats, in human or animal subjects, at least one of cardiac pathologies, vascular pathologies, endocrine pathologies associated with anomalies in hormone secretion, muscle pathologies and/or pathologies of the retina, modulates the activity of the potassium channel activated by polyunsaturated fatty acid and riluzole, and is represented by SEQ ID No. 2 or a functionally equivalent derivative of the sequence, with cells expressing the potassium channel;
measuring effects of the substance on potassium channel transport activity; and
identifying the substance based on the measured effects.

2. The method of claim 1, wherein the polyunsaturated fatty acid is arachidonic acid.

3. A method of diagnosing, in a human or animal subject, a cardiac disease, a vascular disease, an endocrine disease associated with anomalies in hormone secretion, a muscle disease and/or a pathology of the retina involving a mechanosensitive potassium channel represented as SEQ ID No. 2, a gene coding the channel or a functionally equivalent derivative of the sequence and activated by polyunsaturated fatty acid and riluzole comprising:
contacting a biological sample from the subject with an antibody or a mixture of antibodies against said potassium channel; and
detecting the presence or absence of the mechanosensitive potassium channel in the sample.

4. The method of claim 3, wherein the polyunsaturated fatty acid is arachidonic acid.

5. The method according to claim 3, wherein nucleic acids contained in the sample are contacted with one or more nucleotide probes capable of hybridizing with a nucleic acid molecule coding the potassium channel or a functionally equivalent derivative thereof.

6. The method according to claim 3, further comprising determining in the genome of cells present in the sample and localization of a gene coding the mechanosensitive potassium channel.

7. An isolated and purified nucleic acid molecule comprising at least one sequence coding for a protein constituting the hTRAAK, the amino acid sequence of which is SEQ ID NO: 2 or a functionally equivalent derivative of the sequence.

8. The nucleic acid molecule according to claim 2, wherein the sequence is SEQ ID NO: 1.

9. A vector containing the nucleic acid molecule according to claim 7.

10. A vector comprising the nucleic acid molecule of claim 8.

11. A cell transformed with the vector of claim 9, which cell is selected from the group consisting of prokaryotes and eukaryotes.

12. The transformed cell of claim 10 which is a yeast, insect cell, plant cell or mammation cell.

13. The transformed cell of claim 10 which is a bacterium.

14. A method for expression and isolation of a potassium transport channel encoded by a nucleic acid molecule according to claim 1 in a competent host cell comprising transferring a vector including said nucleic acid molecule into a competent host cell, culturing said host cell under conditions allowing the production of the potassium transport channel, and isolating and purifying the polypeptide comprising the potassium transport channel.

15. A pharmaceutical composition for treating and/or preventing at least one of cardiac pathologies, vascular pathologies, endocrine pathologies associated with anomalies in hormone secretion, muscle pathologies and/or pathologies of the retina in humans or in animals, comprising nucleic acids according to claim 7.

16. A method of preventing or treating at least one of cardiac pathologies, vascular pathologies, endocrine pathologies associated with anomalies in hormone secretion, muscle pathologies and/or pathologies of the retina in humans or in animals comprising administering a therapeutically effective amount of the pharmaceutical composition according to claim 15.

17. Procedure for screening substances capable of preventing or treating, in human or animal subjects, cardiac pathologies, vascular pathologies, endocrine pathologies associated with anomalies in hormone secretion, muscle pathologies and./or pathologies of the retina, characterized in that said substances are capable of modulating the activity of the potassium channel activated by polyunsaturated fatty acids, especially arachidonic acid, and by riluzole, the sequence of which is represented in the attached listing as SEQ ID No. 2.

18. Procedure according to claim 17, characterized in that variable quantities of a substance to be tested are brought into contact with cells expressing said potassium channel, and then the possible effects of said substance on the currents of said channel are measured by any suitable means.

19. Procedure for the diagnosis, in a human or animal subject, of a cardiac disease, a vascular disease, an endocrine disease associated with anomalies in hormone secretion, a muscle disease and/or a pathology of the retina which could involve a mechanosensitive potassium channel activated by polyunsaturated fatty acids, especially arachidonic acid, and by riluzole, characterized in that one determines in a biological sample from a patient the presence or absence of a mechanosensitive potassium channel the sequence of which is represented in the attached list as SEQ ID No. 2 or the gene coding this channel or a variant thereof.

20. Procedure according to claim 19, characterized in that said sample is brought into contact with an antibody or a mixture of antibodies against said potassium channel.
